(19) United States
(12) Patent Application Publication
(10) Pub. No.: US 2018/0271559 A1
(43) Pub. Date: Sep. 27, 2018

(54) DEVICES AND METHODS FOR EXFOLIATING AND SANDING

(71) Applicant: JASON BERGER, TRUCKEE, CA (US)

(72) Inventor: JASON BERGER, TRUCKEE, CA (US)

(21) Appl. No.: 15/465,432

(22) Filed: Mar. 21, 2017

(51) Int. Cl. A61B 17/54; A45D 29/00; A61B 17/32

(52) U.S. Cl. CPC A61B 17/54; A45D 29/00; A61B 2017/320008; A61B 2017/320004; A61B 17/32

(57) ABSTRACT

A multilayer material with an outside abrasive layer wrapped around a hollow cylindrical tube that may be used for sanding or exfoliating. The multilayer abrasive material is a continuous roll that is inserted inside the hollow cylindrical tube, wherein a leading edge of the material extends from inside the tube to outside the tube via an elongated slot in an upper part of the body of the tube. The lower part of the body of the tube is a knurled handle. The multilayer material is comprised of a sticky side that adheres to the outside of the tube, and an abrasive side that may be used for sanding or exfoliating. A user may hold the handle of the tube, and use the upper portion of the tube, with the multilayer material attached, to sand or exfoliate, as needed.

### CROSS REFERENCE TO RELATED APPLICATION

This application claims the benefit of pending U.S. provisional patent application No. 62/311,554, filed on Mar. 22, 2016, which is hereby incorporated by reference in its entirety.

### BACKGROUND

This present invention relates to devices and methods for sanding or exfoliating purposes including the sanding of calluses or other items, and exfoliating skin. Often, climbers, cross-fit practitioners, runners, and others develop calluses that must be maintained, smoothed, or removed. The sanding and exfoliating invention disclosed herein provides solutions to problems inherent in the related art.

### SUMMARY

The invention provides a multilayer abrasive material wrapped around a hollow cylindrical tube that may be used for sanding or exfoliating. The multilayer abrasive material is a continuous roll inserted inside the hollow cylindrical tube, wherein a leading edge of the material extends from inside the tube to outside the tube via an elongated slot in an upper part of the body of the tube. The lower part of the body of the tube is a knurled handle.

The multilayer material is comprised of a sticky side that adheres to the outside of the tube, and an abrasive side that may be used for sanding or exfoliating. The multilayer material may be replaced as needed by removing the existing section of material, pulling a new section of the material from inside the cylindrical tube, and sticking the material to the outside of the tube.

A user may hold the handle of the tube, and use the upper portion of the tube, with the multilayer material attached, to sand or exfoliate, as needed.

The present invention is described using the following examples, which may describe more than one relevant embodiment falling within the scope of the present invention.

### BRIEF DESCRIPTION OF DRAWINGS

FIG. 1 is a perspective view of an embodiment of the invention, showing the cylindrical tube without the multilayer material.

FIG. 2 is a perspective view of an embodiment of the invention, with the end caps removed.

FIG. 3 is perspective view of an embodiment of the invention, with a leading edge of the multilayer material extending out from the elongated slot, and showing the upper abrasive side of the multilayer material. The dotted lines represent the roll of multilayer material inside the cylindrical tube.

FIG. 4 is perspective view of an embodiment of the invention, with a leading edge of the multilayer material extending out from the elongated slot, and showing the bottom side of the multilayer material.

FIG. 5A is a perspective view of an embodiment of the invention, showing a segment of the multilayer material torn off from the roll, and adhered to the outside of the cylindrical tube.

FIG. 5B is a perspective view of an embodiment of the invention, showing a segment of the multilayer material still connected with the roll, and adhered to the outside of the cylindrical tube.

FIG. 6 is a plan view of the layers of the multilayer material.

FIG. 7 is a flow chart of an embodiment of methods of using the invention.

### DETAILED DESCRIPTION

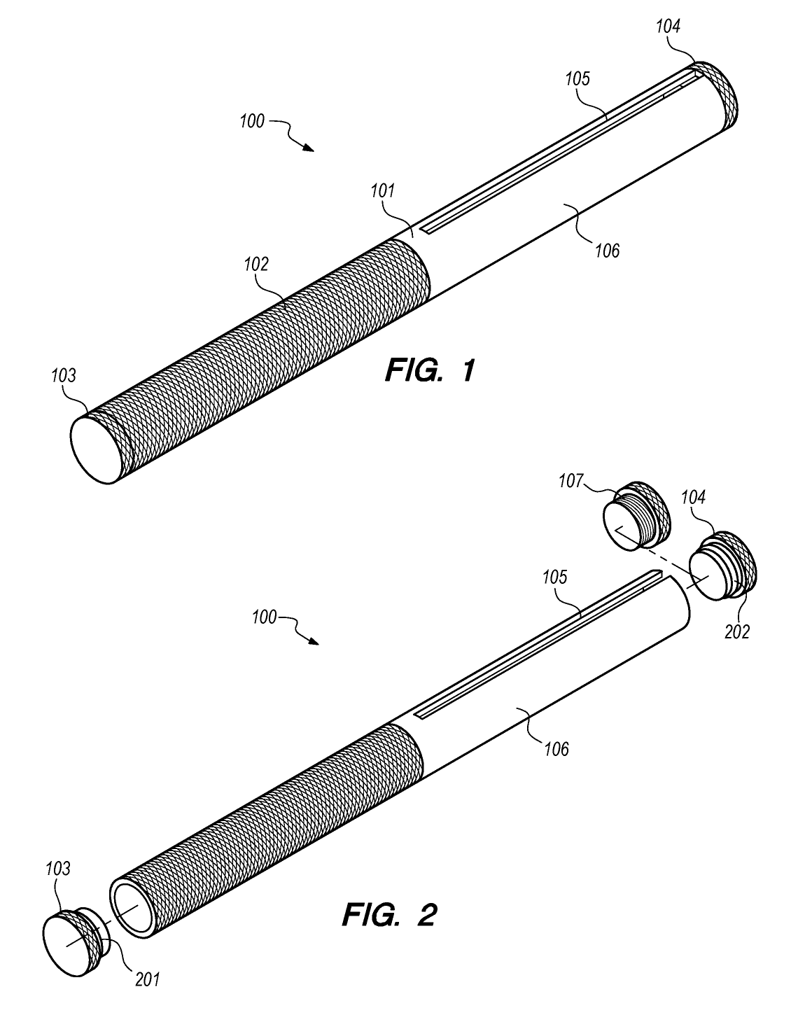

Referring now to FIG. 1, a sanding tool 100 is provided. Sanding tool 100 has a cylindrical tube 101. Cylindrical tube 101 is hollow and open at opposing ends. Cylindrical body 101 may be manufactured of a durable and workable material such as aluminum, stainless steel, polymer or any suitable material. Cylindrical body 101 includes a handle portion 102 where the body is gripped by a user during the process of sanding or exfoliating. The handle portion of body 101 is knurled about the circumferential surface of the device from one end to a length on or around the mid-point of the device in order to facilitate a good grip of the device by a user.

Cylindrical body 101 includes an elongate slot 105 placed through the cylinder wall along the length of the material staging portion of the tool body 106, the slot extending through the cylinder wall and open at the end of cylindrical tube 101. Slot 105 has a width dimension larger than the thickness dimension of the multilayer material 300.

Referring now to FIG. 2, device 100 is depicted herein with first cap 103 and second cap 104 removed. In this embodiment, cap 103 and cap 104 share the same design although this is not required to practice the present invention. Caps 103 and 104 have a major outside diameter that is equal to or just larger than the outside diameter of the cylindrical body 101 of tool 100. First cap 103 and second cap 104 have a minor outside diameter that is just smaller that the inside diameter of the cylindrical body of tool 100.

In a preferred embodiment, cylindrical body 101 has a first removable cap 103 to cover the handle end of the device and a second removable cap 104 covering the opposite end of the device. In one embodiment, cap 103 and cap 104 are also knurled to facilitate good grip in removal and replacement of those caps during use. Caps 103 and 104 may be identical parts and are, in this embodiment, press fitted into the open ends of the cylinder.

In some embodiments, both caps 103 and 104 may be press fitted onto the ends of cylindrical body 101. In these embodiments, cap 103 and cap 104 both include a peripheral groove 201 placed laterally about the minor outside diameter of the cap proximal to the knurled major diameter boundary and the inserted edge of the cap. The grooves support a first O-ring gasket 202 seated in groove on cap 103, and a second O-ring gasket 202 seated in groove on cap 104. In these embodiments, the outside diameter of first and second O-rings 202 is just larger than the inside diameter of the cylindrical body to facilitate a press fit for both caps. In this way, caps 103 and 104 may be pulled off and pressed back onto the cylindrical body 101 of sanding tool 100. O-rings may be made of rubber or a polymer or another resilient and flexible or semi-flexible material.

In other embodiments, caps 103 and 104 may be threaded 107 and sized to screw into threaded ends of cylindrical body 101.

In yet other embodiments, there may be only one cap 104, connected with the material staging portion of the cylindrical tube.

In one embodiment, shown in FIG. 2, slot 105 is open at the end of the cylindrical body, so that slot 105 has two long edges and a short edge connecting them. In other embodiments, slot 105 may be closed at the end of cylindrical body 101, so that slot 105 has four sides or edges.

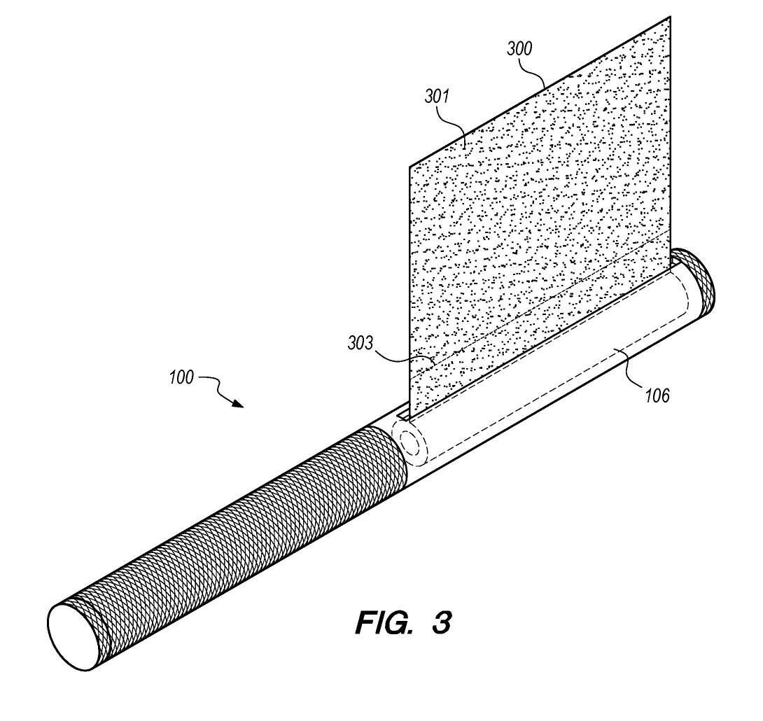

Referring now to FIG. 3, an abrasive medium is provided in the form of a length of a multilayered material 300 rolled into a roll of material that fits inside cylindrical body 101. The roll of abrasive medium has a leading outside edge that is “trained” or directed into and through slot 105 such that the roll may be pulled out through slot 105 a particular segment length enabling wrap-around of the material 300 over cylindrical body 101. In some embodiments, a lateral perforation line 303 or “cut line” may be provided in the material to define tear-off points along the length that result in a correct segment of material 300 relative to wrapping the segment of material about the cylindrical body 101 with little or no overlap of material 300. In other embodiments, there may be no lateral perforations, and the user may tear or cut material 300 to the desired length.

Material 300 is a multilayered abrasive material having an overall length rolled up inside of tool 100 and a width (roll width) just smaller than the length of slot 105 enabling the leading edge of material 300 to be inserted from the inside of hollow cylindrical body 101 through slot 105, as depicted in FIG. 3.

Material 300 has an outside or top abrasive layer 301 that may have any level of abrasiveness desired. In a preferred embodiment the abrasiveness is as needed for exfoliating or sanding calluses. In other embodiments, the device may have a relatively low abrasiveness for polishing or buffing skin. In yet other embodiments, the device may have high abrasiveness for sanding materials other than skin, as a non-limiting example wood.

As shown in FIG. 6, multilayer material 300 comprises a layer of a gel backing material 600 under abrasive layer 301, wherein the gel layer 600 is resilient and flexible. Gel layer 600 is in between adhesive layer 400 and abrasive layer 301. Adhesive layer 400 has sufficient adhesive to securely stick or adhere multilayer material 300 to the outside circumference of device 100, and is adapted to provide a retention of the multilayered material to the outside surface of the tool when the material is wrapped about the tool leaving the abrasive surface facing outward. Adhesive layer 400 simultaneously has sufficient adhesive to allow for relatively easy removal of material 300 after use. Multilayer material 300 further includes an inside protective peel-off layer 401 that may be peeled off to expose adhesive layer 400.

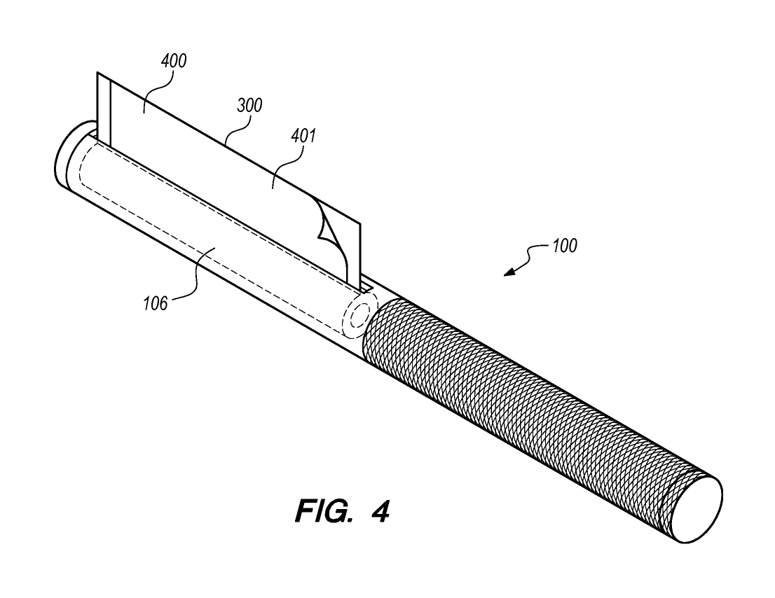

Referring now to FIG. 4, sanding tool 100 is rotated about in this view to depict the inside adhesive surface 400 of multilayer material 300. Adhesive layer 400 may be protected by a peel-off paper 401 to protect the adhesive surface until a user is ready to adhere the material to the surface of the sanding tool. Peel-off paper 401 may be wax paper, or other non-stick paper. The width dimensions of the abrasive layer 301 and gel layer 600 are generally the same. The width dimensions of the adhesive layer 400 and non-adhesive peel-off layer 401 are approximately the same also.

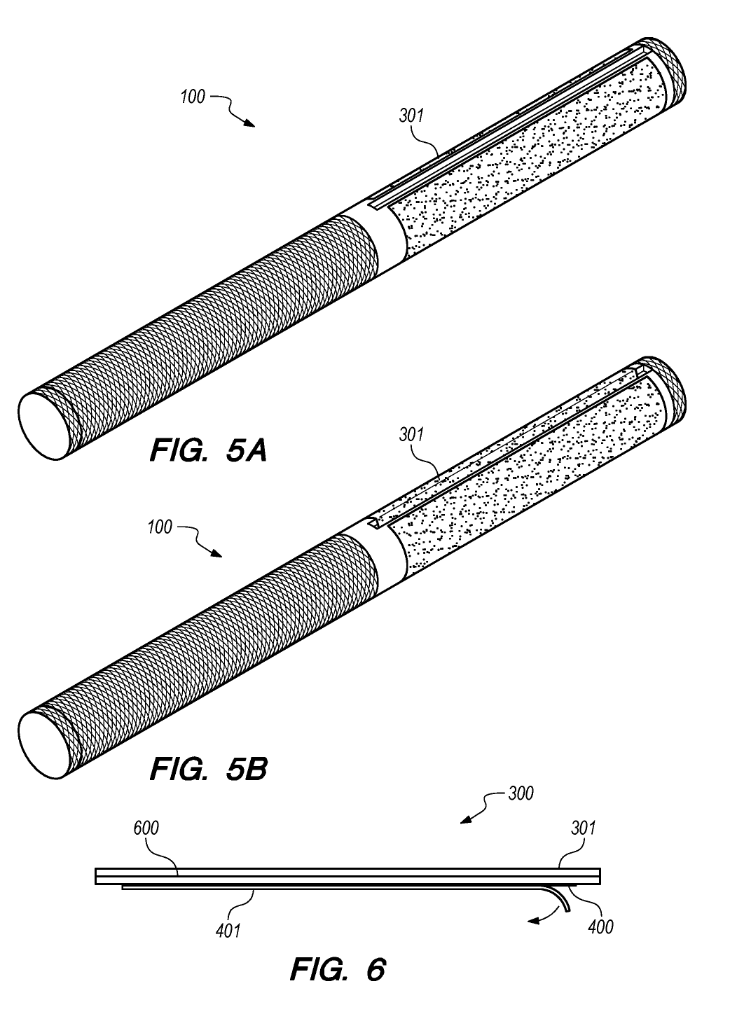

Referring now to FIG. 5A, device 100 is depicted in this example with the multilayer material 300 wrapped about the material staging portion 106 of tool 100 with the abrasive layer 301 facing outward to produce a cylindrical sanding or exfoliating tube. As shown in FIG. 5A, a user may retrieve a length of multilayer medium 300 through slot 105 and tear it off at a manufactured perforation line 303 that demarcates each segment of sanding medium 300 in the roll of material inside the tool, and wrap this torn-off segment around the outside circumference of the cylindrical tube.

As shown in FIG. 5B, in another embodiment a user may retrieve a length of multilayer medium 300 through slot 105 and instead of tearing it off, the user may allow the multilayer material to remain connected with the roll, and may wrap a connected segment around the outside circumference of the cylindrical tube.

In a preferred embodiment, the multilayer material section 300 is wrapped about the sanding tool staging portion 106 one time with very little or no overlap of material 300. In other embodiments, it is apparent that material 300 may overlap.

The sanding tool may be used until the abrasive layer 301 is worn to a point where a new section of abrasive medium may be required for further use. The wrapped section may be peeled off from the outside circumference of device 100 and a new multilayer material section may be retrieved from inside the tool through slot 105, and wrapped around the outside of the cylindrical tube.

In a one embodiment the user may, if desired, remove cap 104 to have access to the inside material roll to urge or replace the leading edge of the multilayer material 300 back through the slot, retrieving the leading edge through slot 105 up to the next perforated line 303 to tear off another segment for a next use. In some embodiments of the invention, there may be more than one roll of multilayered material stored within the cylindrical body of the device. Device 100 may vary in length and in diameter without departing from the spirit and scope of the present invention. The dimensions of the material roll are guided by the dimensions of the sanding tool relative to the inside diameter of the cylindrical body and the slot length accessible with the cap on.

Referring now to FIG. 6, multilayer medium 300 has abrasive layer 301 as an exposed top layer. Abrasive layer 301 is backed by a flexible solid gel layer 600. Gel layer 600 provides the body of multilayer medium 300. Abrasive material 301 may be sprayed or coated onto, or otherwise connected, or fused to one side of gel layer 600. Multilayer medium 300 includes an adhesive, or sticky layer 400. Adhesive later 400 may be connected with the other side of gel layer 600, opposite abrasive material 301. Adhesive layer 400 may be sprayed, coated, or otherwise fused to the gel back surface. Adhesive layer 400 allows for a firm hold onto the surface of the tool such that the abrasive material 301 may be used vigorously without sliding about on the tool. The adhesive layer 400 also allows the abrasive medium 301 to conform to (and stay conformed to) the shape of the sanding tool.

Multilayer material 300 includes a peel off protective paper backing 401 that may be a type of wax paper, or other thin flexible material, that protects sticky layer 400 from exposure and contact with other components until ready for use. As illustrated in FIG. 6, backing material 401 may be peeled off in the general direction of the arrow. Adhesive layer 400 is adapted to stick to the staging surface 106 of the sanding tool but not so much so that the material becomes stuck to the tool. The properties and tensile strength of the abrasive material section are such that it may be easily peeled off of the tool in one section after use.

After using the tool 100 such that the abrasive material 301 is worn away, a user may simply peel off the old multilayer material 300 and replace it with a new segment of multilayer material 300 from the roll inside the device.

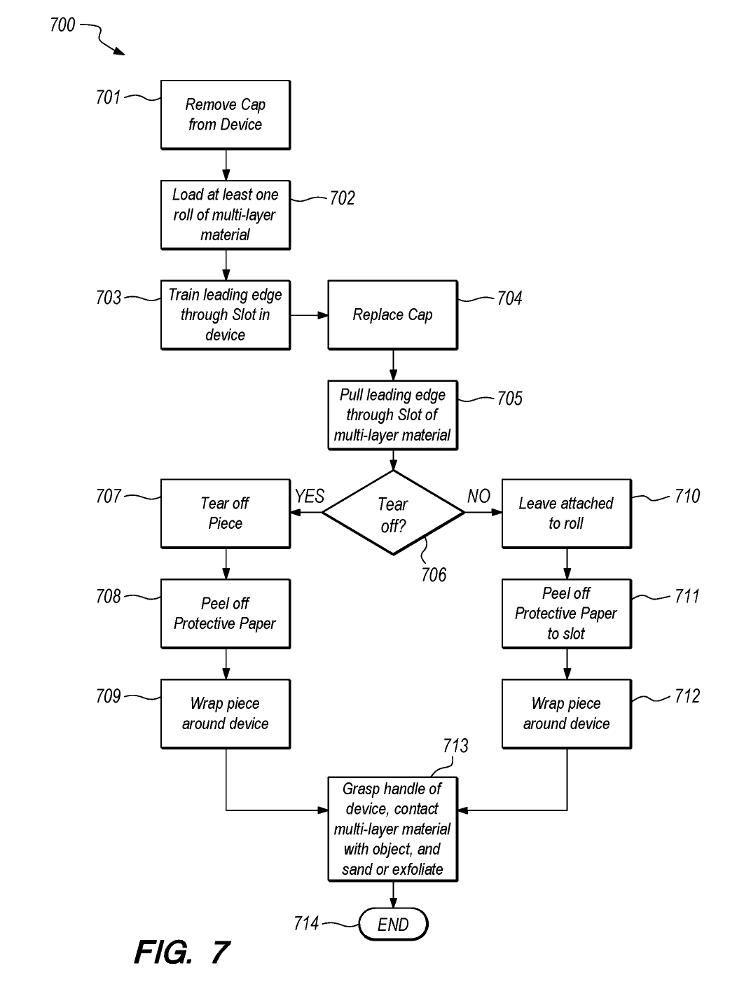

Referring now to FIG. 7, a process 700 is provided for staging multilayer material 300 about the tool 100 for use. At step 701 a user may remove the cap 104 from the staging area portion 106 of the tool containing slot 105 in order to load 702 at least one roll of multilayer sanding material 300, as shown in FIG. 3. At step 703, the user may urge, direct, or train the leading edge of the roll through slot 105 from the inside of cylindrical body 101, through slot 105, to the outside of cylindrical body 101. It is noted herein that the material roll requires no orientation when loading into the sanding tool.

At step 704 the user may replace the cap. At step 705, the user may pull out the leading edge of the multilayer material 300 through slot 105. In one embodiment, multilayer material 300 has periodic perforations 303 separating material 300 into segments where the length of each segment is equal to the outside circumference of the cylinder. In this embodiment the user extends material 300 out until a first lateral perforation line 303 is visible. At step 706 the user may tear off a segment of material 300 at the perforation. In one embodiment, the user utilizes an edge of slot 105 in the sanding tool to tear 707 material 300, whether or not there are perforations 303 in material 300. With a section torn off at step 706, the length of the segment is just long enough to wrap around the circumference of staging area 106 of the sanding tool.

In another embodiment, the user may extend material 300, and simply adhere it to the outside circumference 106 of the cylindrical tube, without separating the material 710 from the roll. The rest of the roll of material remains inside the sanding tool.

At step 708 or step 711, the user may peel off the protective backing material 401 off of the sticky layer of the material. At step 709 or 712, the user may wrap the multilayer material segment lengthwise about the staging area 106 circumference of the tool body. The product is at that point staged for use in sanding calluses or for sanding anything else, as desired or needed. The user may grasp 713 the handle, and place the abrasive layer in contact with a callus, or other material to be sanded. The process may end at step 714. The invention may be used and reused for sanding until the abrasive is worn beyond usefulness at which point the user may peel off the used material and repeat process 700 to stage the device for a next use period.

### CONCLUSIONS, OTHER EMBODIMENTS, AND SCOPE OF INVENTION

The above description presents the best mode contemplated in carrying out the invention(s). However, it is susceptible to modifications and alternate constructions from the embodiments shown in the figures and accompanying description. Consequently it is not intended that the invention be limited to the particular embodiments disclosed. On the contrary, the invention is intended to cover all modifications, sizes and alternate constructions falling within the spirit and scope of embodiments of the invention.

It will be apparent to one with skill in the art that the callus removal apparatus and abrasive material medium of the invention may be provided using some or all of the mentioned features and components without departing from the spirit and scope of the present invention. It will also be apparent to the skilled artisan that the embodiments described above are specific examples of an inventive concept which may have greater scope than any of the singular descriptions taught. There may be many alterations made in the descriptions without departing from the spirit and scope of the present invention.

The above description presents the best mode contemplated in carrying out the invention(s) described herein. However, it is susceptible to modifications and alternate constructions from the embodiments shown in the figures and accompanying description. Consequently it is not intended that the invention be limited to the particular embodiments disclosed. On the contrary, the invention is intended to cover all modifications, sizes and alternate constructions falling within the spirit and scope of embodiments of the invention.

It will be apparent to one with skill in the art that the callus removal apparatus and abrasive material medium of the invention may be provided using some or all of the mentioned features and components without departing from the spirit and scope of the present invention. It will also be apparent to the skilled artisan that the embodiments described above are specific examples of an inventive concept which may have greater scope than any of the singular descriptions taught. There may be many alterations made in the descriptions without departing from the spirit and scope of the present invention. What is claimed is:

1-10. (canceled)

11. A device for sanding or exfoliating comprising:
a hollow cylindrical tube with an outside circumference wherein a length from the first end of the outside circumference is smooth and functions as a staging area for a multilayered abrasive material, and wherein a length from second end of the outside circumference is knurled and functions as a handle;
a roll of multilayered abrasive material, wherein the multilayered material has a length, width, and a thickness, the multilayered material is rolled along its length and disposed within the hollow cylindrical tube in proximity to the staging area of the tube, the length dimension supporting continuous use of the abrasive material by applying, retiring, and restaging segments thereof over the life of the material roll;
an elongated slot disposed through the wall of the cylindrical tube, disposed within the staging area and near the material roll, the slot defined by two substantially parallel long edges extending along a length of the cylindrical tube from the first end, the slot terminating at a strategic point wherein the length and width of the slot are sufficient to receive therethrough, the width and thickness of a leading edge the material roll.

12. The device of claim 11, wherein the multilayered abrasive material includes a removable protective layer covering an adhesive layer, the adhesive layer covering a gel layer that the abrasive material is coated over or otherwise disposed thereon.

13. The device of claim 12, wherein a segment of the multilayered abrasive material may be extended from the roll inside the cylindrical tube, through the elongated slot and to the outside of the tube, and wherein the protective layer may be removed, the segment may be wrapped about the circumferential surface of the cylinder and adhered to the circumferential surface by way of the adhesive layer, and staged thereto, the abrasive layer facing outward.

14. The device of claim 11 wherein there is a second removable cap sized to securely fit inside the second end of the cylindrical tube.

15. The device of claim 11 wherein the removable cap comprises a minor outside diameter that is just smaller that the inside diameter of the cylindrical tube, a peripheral groove sized to receive an O-ring, and an O-ring, wherein the cap may be pressed into the end of the cylindrical tube.

16. The device of claim 11 wherein the removable cap comprises a threaded minor outside diameter that is just smaller than a threaded inside diameter of the cylindrical tube, wherein the cap may be screwed into the end of the cylindrical tube.

17. The device of claim 12 wherein the segments are marked by perforations in the multilayered material, and wherein each segment is substantially equal in length to the outside circumference of the staging area of the cylindrical tube.

18. The device of claim 17 wherein a segment of multilayer material is torn from the roll, and is adhered via the adhesive layer to the outside circumference of the staging area of the cylindrical tube.

19. A method of exfoliating or sanding skin, using a sanding device having a hollow cylindrical tube with an outside circumference wherein a length from the first end of the outside circumference is smooth and functions as a staging area for a multilayered abrasive material, and wherein a length from second end of the outside circumference is knurled and functions as a handle, a roll of the multilayered abrasive material, wherein the multilayered material has a length, width, and a thickness, the multilayered material rolled along its length and disposed within the hollow cylindrical tube in proximity to the staging area of the tube, an elongated slot disposed through a wall of the cylindrical tube, with the staging area and near material roll, the slot defined by two substantially parallel long edges extending along a length of the cylindrical tube from the first end the slot terminating at a strategic point wherein the length and width of the slot is sufficient to receive therethrough the width and thickness of a leading edge the multilayered abrasive material, the method including steps for:
training a leading edge of the multilayered abrasive material from the roll inside the cylindrical tube, through the elongated slot, and to the outside of the cylindrical tube;
extending the multilayer material to a segment equal to the outside circumference of the staging area of the cylindrical tube;
removing the protective layer of the multilayer material;
adhering the multilayer material to staging area by connecting the adhesive layer to outside circumference of the staging area of the cylindrical tube so that the abrasive layer faces outward;
grasping the knurled handle;
positioning the device so that the abrasive layer connects with a callus; and
sanding the callus by moving the device so that the abrasive layer rubs the callus.

20. The method of claim 19, wherein the segment of the abrasive material is predefined by perforations on the material and is torn off segment by segment.

21. The method of claim 19, wherein the segment is staged and applied about the cylinder but remains contiguously connected to the roll within the cylinder until the segment is expired.

22. The method of claim 19, wherein after the abrasive layer is worn down, the multilayered material is removed from the staging area of the cylindrical tube, and wherein a next segment of multilayered material is urged out and around the outside circumference of the staging area of the cylindrical tube.

23. The device of claim 11, wherein one or both of the long edges defining the elongated slot is a cutting edge having a sharp profile sufficiently sharp for cutting through the multilayered abrasive material.
